(19) United States
(12) Patent Application Publication
(10) Pub. No.: US 2016/0103073 A1
(43) Pub. Date: Apr. 14, 2016

(54) FLUORESCENCE REMOVAL FROM RAMAN SPECTRA BY POLARIZATION SUBTRACTION

(71) Applicant: Alakai Defense Systems, Inc., Largo, FL (US)

(72) Inventors: Alan R. Ford, Saint Robert, MO (US); Adam J. Hopkins, Petersburg, FL (US)

(21) Appl. No.: 14/883,269

(22) Filed: Oct. 14, 2015

Related U.S. Application Data
(60) Provisional application No. 62/063,472, filed on Oct. 14, 2014.

(51) Int. Cl. G01N 21/65; G01N 33/22

(52) U.S. Cl. CPC G01N 21/65; G01N 33/227; G01N 2201/06113; G01N 2201/121; G01N 2201/0683

(57) ABSTRACT

A method for utilizing polarization as a scheme for fluorescence removal from UV Raman spectra collected in a standoff detection scheme has been invented. In this scheme, a linearly polarized ultraviolet (UV) laser interacts with a material on a surface or in a container. The material generates Raman scattering with polarization contributions relative to that of the laser. The material possibly fluoresces as well, but the fluorescence is generally unpolarized. By subtracting a scaled version of the perpendicular component from the parallel component of the returned signal both relative to the laser source polarization—it is possible to generate a spectrum that is fluorescence free and contains the strongest features of the Raman scattered light.

### RELATED APPLICATIONS

This application claims, under 35 U.S.C. §119 and/or §120, priority to and the benefit of provisional application U.S. Ser. No. 62/063,472 filed Oct. 14, 2014, which is incorporated by reference in its entirety herein.

### BACKGROUND OF THE INVENTION

1. Field of the Invention

The present invention relates to apparatus, systems, and techniques for processing a Raman scattering signal and, in particular, to distinguishing Raman spectra from fluorescence in Raman spectroscopy, including when the Raman spectra and fluorescence is generated by and detected at an instrument at standoff distances from a target material.

2. Problems in the Art

When applying Raman spectroscopy to the detection of explosives or toxic substances at standoff ranges (e.g. meters to tens of meters, and perhaps one hundred meters and possibly more), a chief drawback is the obscuring of the Raman scattering signal by fluorescence.[1,2] Fluorescence is generally excited whenever a laser in the ultraviolet (UV), visible, or near-visible portion of the electromagnetic spectrum is used for Raman spectroscopy. The fluorescence quantum yields of most materials are generally orders of magnitudes greater than the scattering through the Raman effect. [3] In addition, the Raman signal is largest for UV wavelengths, which also induces the largest excitation of fluorescence. Consequently, the fluorescence can saturate a detector to the point that the Raman scattering is too insignificant to detect, especially in the UV.

Because of the desire to use UV wavelengths for Raman detection, several strategies to avoid fluorescence have been proposed. Using a laser with a wavelength (λ) less than 250 nm allows the Raman spectrum of most materials to be collected in the spectral region between the laser wavelength and the Stokes shifted fluorescence of almost all known materials. [4] Although this strategy improves the Raman signal intensity greatly and avoids most of the fluorescence from the sample and background in the useful Raman spectrum, it introduces the possibility of photodegrading the sample.[5,6] In addition, powerful UV lasers with λ<250 nm for applications such as standoff detection are limited to excimer sources, which are less than ideal. Collection of UV Raman spectra at different wavelengths and subtracting the spectra to remove fluorescence contributions (i.e. Shifted-Excitation Raman Differential Spectroscopy or SERDS) [7] is alternative method to mitigating fluorescence, but it requires at least some ability to tune the laser source. Finally, software approaches to remove potential baselines [8] can also be used provided that the fluorescence does not completely mask the Raman signal, which is a real possibility when UV wavelengths are used.

Another strategy for rejecting fluorescence as well as improving identification in Raman spectroscopy is similar to SERDS but uses polarization rather than wavelength shifts of the source. Raman scattering is known to depend on the linear polarization of the collected spectrum relative to the linear polarization of the excitation source.[10] Molecular vibrations that are highly symmetric tend to return more light of the same polarization as the excitation source (denoted I_{∥}) whereas asymmetric vibrations are less likely to do so, as monitored by filtering perpendicular to the source (denoted Î).[10] However, the fluorescence has virtually no dependence on the polarization of the source, except in a few cases that are generally applicable for viscous liquids.[11] Using this fact, “depolarization” spectra can be obtained, [12] as in FIG. 1, and depolarization ratios (r) can be created where Î is ratioed to I_{∥}. Such a technique has been employed to some extent by Edgewood Chemical and Biological Center (ECBC) in the near-IR regime for fluorescence and Raman-scattered background rejection in the detection of toxic materials (FIG. 1) and explosives.[13] However, this was taken in a laboratory setting, and used near an IR source.

As can be seen, there are different approaches to using Raman information for identification of constituent elements in an unknown sample. It can also be seen that there are a number of factors, some of which are antagonistic to one another, that are involved. The inventor has identified there is room for improvement in this technical field.

### SUMMARY OF THE INVENTION

Although the polarization dependence of Raman has been known for some time, and a recent technique subtracting the two polarized Raman spectra has been proposed, the present invention applies it in a different way. By subtracting rather than ratioing the UV Raman scattering at polarizations parallel and perpendicular to the excitation source, a UV Raman spectrum can be generated that is almost entirely free of fluorescence, with only a small loss in signal—at most a factor of 4 in theory—for UV Raman transitions. Le Ru et al. [3] have applied the technique for the extraction of Raman cross-sections, but not using UV wavelengths. Their demonstration of the method involved visible wavelengths for excitation where the fluorescence is generally worse for Raman. Moreover, their analysis of a visible dye with this method showed how powerful it can be at rejecting the fluorescence in favor of the Raman spectrum. It should be noted however that this technique works best for Raman transitions that are highly symmetric,[13] as the parallel component polarization will be strongest.

One major advantage of the use of this polarization technique as well as depolarization ratios is that UV Raman spectroscopy could be acquired using wavelength sources such as Nd³⁺:YAG lasers that are cheaper, easier to maintain, and more rugged. Inherently-polarized Q-switched Nd³⁺:YAG lasers at 266 nm and 355 nm with high power (>50 mJ/pulse) and/or high repetition rates are desirable for Raman. Polarization methods have already been used to extract UV Raman spectra using these types of lasers under conditions of high fluorescence in flames where acquiring the Raman spectra at different polarizations allowed for discrimination of the signal from the highly-emissive background.[14] However, they have not been applied for detection of such things as explosives.

Raman standoff detection of explosives would be one of the greatest beneficiaries by the application of these techniques with such laser sources. For example, using a polarized deep UV (DUV) laser at say 248 nm, and flipping between parallel and perpendicular at some rate (say 2 Hz) at a receiver, (see, e.g., FIG. 3), a significant standoff detection performance improvement over current DUV Raman spectroscopy without using polarization methods may be obtained. Using a polarized DUV (260-266 nm) solid-state laser in a similar instrument may likewise offer a significant reduction in fluorescence background, sufficient to get as good performance using these wavelengths as currently possible with 248 nm sources for at least some explosives. If the method only reduces the fluorescence noise by ˜10×, it would allow a more ruggedized, compact laser to be used rather than excimer sources that are currently the only viable option for standoff Raman spectroscopy with sources below 250 nm. In a similar way, a polarized UV (320-360 nm) laser in a near-identical instrument may provide even further reductions to the fluorescence noise (which would be worse at these wavelengths), while also allowing a much more ruggedized, compact laser than an excimer. An additional advantage, provided that the fluorescence can be reduced by >>10×, is that eye safe operations at 300× higher laser power (300× more signal) than can be used at DUV wavelengths below 250 nm are possible.

In one aspect, the present invention comprises utilizing polarization as a scheme for fluorescence removal. In this scheme, a linearly polarized ultraviolet (UV) laser interacts with a material on a surface or in a container. The material generates Raman scattering and possibly fluorescence. The fluorescence is generally unpolarized, but the Raman scattering depends on the polarization of the laser and the symmetry of the normal modes in a material. By placing a polarized filter in front of a detector, it is possible to measure the components of the Raman scattering that are parallel and perpendicular to the polarization of the laser. Both these components will contain approximately equal amounts of the fluorescence generated by the laser target. By subtracting a scaled version of the perpendicular component from the parallel component, it is possible to generate a spectrum that is fluorescence free and contains the strongest features of the Raman scattered light. This technique can take on a number of embodiments when implemented in practice.

In one embodiment of this invention, the analyzed material is a solid, liquid, gas, or mixture of states.

In one embodiment, the analyzed material is a mixture of chemicals.

In one embodiment, the analyzed material is on a surface.

In another embodiment, the analyzed material is in a container.

In certain embodiments of this invention, the laser source is a UV laser with a wavelength between 220 and 400 nm.

In one embodiment, the laser source is a solid-state UV laser.

In one embodiment, the laser source is an excimer.

In one embodiment of this invention, the laser source is pulsed.

In one embodiment of this invention, the laser source is continuous wave (cw) or pseudo-cw.

In one embodiment of this invention, the laser polarization is switched using a polarization filter which is rotated to different orientations.

In one embodiment of this invention, the laser polarization is switched using a fixed polarization filter and a waveplate rotated to different orientations.

In one embodiment of this invention, the laser polarization is switched by inserting one or multiplicity of polarization selective optics.

In one embodiment of this invention, the receiver or collector is a telescope.

In another embodiment of this invention, the receiver is a collection of lenses, mirrors, and related focusing optics.

In one embodiment of this invention, the receiver polarization is switched using a polarization filter which is rotated to different orientations.

In another embodiment, the receiver polarization is switched using a fixed polarization filter and a waveplate rotated to different orientations.

In another embodiment, the receiver polarization is switched by inserting one or multiplicity of polarization selective optics.

In one embodiment, the received light is split into two signals using a beam splitter before passing each through a polarization filter. In one embodiment, the received light is split into parallel and perpendicular polarizations, each of which are simultaneously measured.

In one embodiment of this invention, the polarized Raman spectrum that is perpendicular to the polarization of the laser source is directly subtracted from the polarized Raman spectrum that is parallel to the polarization of the laser source.

In one embodiment of this invention, the polarized Raman spectrum that is perpendicular to the polarization of the laser source is scaled before being subtracted from the polarized Raman spectrum that is parallel to the polarization of the laser source.

In one embodiment of this invention, the polarized Raman spectra are preprocessed before performing spectral combination.

In another aspect of the invention, a hand-held instrument includes a polarized UV laser source to generate an interrogating laser beam to standoff distances, a collector of return light from the interrogation, and a polarizer of the return light that can be adjusted between different polarization states. Spectra of the return light—each polarized in a different polarization state—are produced in a portable spectrometer operatively connected to the hand-held instrument. Those spectra are quantified and compared in a portable computer. The comparison can be used to remove fluorescence and better distinguish Raman information to more accurately detect constituent chemicals in the return light. The hand-held instrument includes structure to allow quick and easy adjustment of the polarizing element between the two polarization states. It can utilize an intrinsically polarized laser or can include another polarizing element in the hand-held instrument and external of the laser source which can be set to one polarization state or optionally adjusted between at least two different polarization states. Utilizing UV laser sources and the adjustable polarization states allows a portable, cost-effective system for standoff distances, including meters to tens of meters for both indoors and outdoors.

### BRIEF DESCRIPTION OF THE DRAWINGS

FIG. 1 is a graph of polarized Raman spectrum of dimethyl methylphosphonate (DMMP) as taken from [13]. The O-P-O bend is very symmetric, giving a much larger return along I_{1∥}.

FIG. 2 is a graph of polarized 262 nm Raman spectrum of a 1.9 M solution of ammonium nitrate in water collected in the deep UV that demonstrates aspects of the invention.

FIG. 3 is a highly diagrammatic view of one embodiment of the technique according to aspects of the present invention. Here, a linearly polarized UV laser illuminates a sample in a cuvette. The scattering from the sample is passed through a lens then through a polarized filter that can select either the parallel or perpendicularly polarized light, after which the light is passed to a spectrometer where the selected component is analyzed.

FIG. 4 is a flowchart of a detection process that could be used with the technique of FIG. 3.

FIG. 5 is a side elevation view with side wall removed to show interior components of one embodiment of an apparatus to implement the technique of FIG. 3, including the source and receiver of an apparatus for performing detection with Raman after fluorescence removal via combination of polarized Raman spectra where the source is intrinsically polarized.

FIG. 6 is similar to FIG. 5, but is an alternative embodiment of the source and receiver of the apparatus for performing detection with Raman after fluorescence removal via combination of polarized Raman spectra where the source is polarized by an external polarizing element.

FIG. 7 is a highly schematic illustration of incoming light being split into individual polarized components for a single spectrometer and detector according to one exemplary embodiment of the invention.

FIG. 8 is similar to FIG. 7, but is an illustration of incoming light being split into individual polarized components for multiple spectrometers and detectors according to one exemplary embodiment of the invention.

FIG. 9 is a diagram of components of polarized light passed by a polarized filter. Rotating the filter passes the other component of polarized light.

FIG. 10 is a diagram of polarized light passing through a waveplate and polarized filter.

FIG. 11 is a diagram illustrating the use of polarization optics that may be inserted into a light path for comparison to FIG. 9 and FIG. 10.

FIG. 12 is a graph showing Raman spectra of dimethyl methylphosphonate (DMMP) using a 262 nm laser as collected by one embodiment of the invention.

FIG. 13 is a graph showing Raman spectra of dimethyl methylphosphonate (DMMP) using a 262 nm laser collected by one embodiment of the invention where the I_{⊥} has been scaled. The result of subtracting I_{∥}−I_{⊥} is also shown.

FIG. 14 is a graph showing the two polarized components and their subtraction result for ammonium nitrate fuel oil (ANFO), according to an aspect of an exemplary embodiment of the present invention. The peak at 1650 cm⁻¹ is believed to be from the fuel oil, while the feature at 1040 cm⁻¹ is from ammonium nitrate. An offset has also been subtracted from I_{⊥}.

FIG. 15 is a flowchart of spectral combination and detection algorithms according to further embodiments of the present invention.

FIG. 16 is similar to FIGS. 5 and 6, an illustration of a man-portable apparatus integrating the fluorescence subtraction technique and including other possible components to create an overall detection system according to aspects of the invention.

FIG. 17 is a schematic diagram of a manually translatable polarizing filter (adjustable between 90° alternative polarization states).

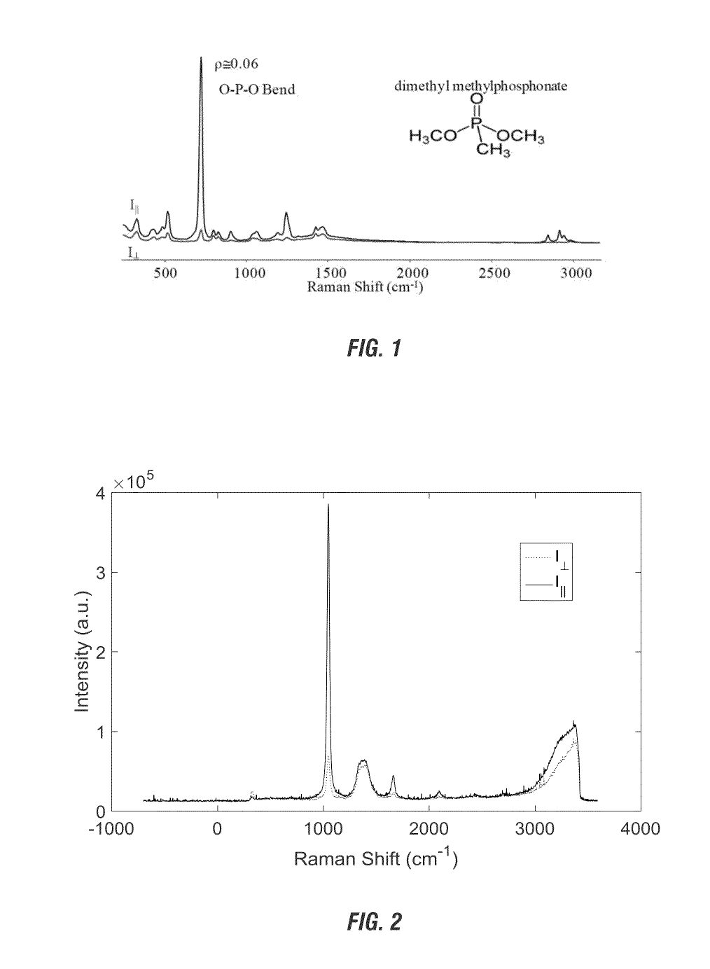

### DETAILED DESCRIPTION OF EXEMPLARY EMBODIMENTS OF THE INVENTION

### Overview

For a better understanding of the invention, specific forms or embodiments it can take will now be described in detail. Frequent reference will be taken to the accompanying drawings, which are itemized above. Reference numerals will be used to indicate certain parts or locations in the drawings. The same reference numerals will indicate the same parts or locations unless otherwise indicated.

These embodiments will focus upon stand-off distance detection of chemical substances with a portable detection system. However, it is to be understood that individual aspects could be implemented in different ways, at different distances, and for different applications.

The examples given are neither inclusive nor exclusive of all the forms and embodiments that aspects of the invention can take.

### Method

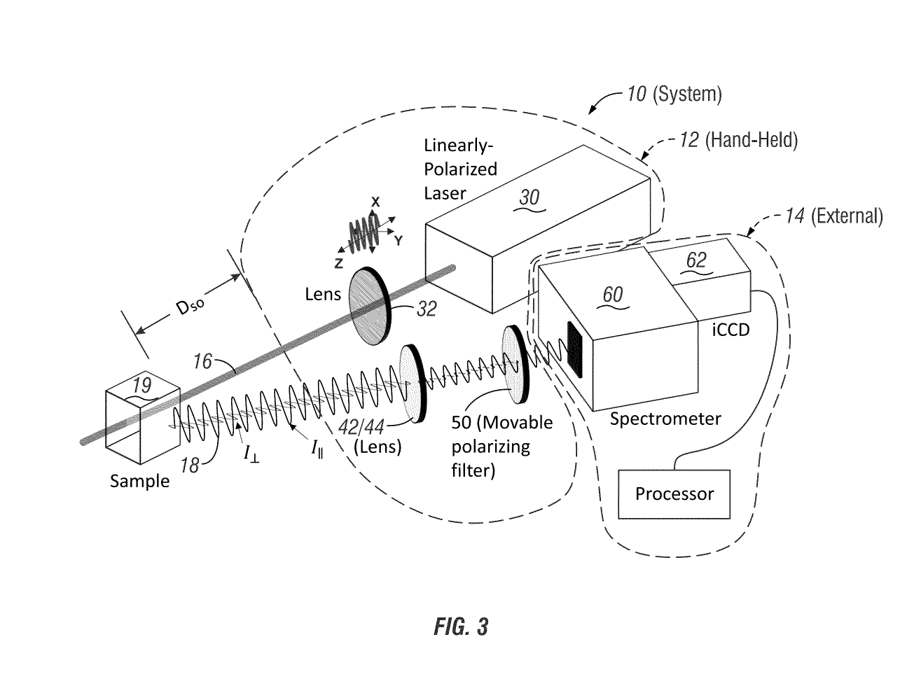

In one aspect or embodiment, the present invention seeks to remove fluorescence from a Raman spectrum collected at a standoff distance (see, e.g., diagrammatically illustrated distance D_{SO} in FIG. 3). By standoff distance it is meant at least not directly adjacent or in abutment from the collector. In most cases, standoff distance D_{SO} would be at least a meter. A linearly polarized laser propagates light on a target located at distances of, e.g., more than a meter from the light source. At the target, fluorescence can be generated from all materials with which the light interacts, but the fluorescence shows little dependence on the polarization of the source. However, the light is scattered into at least two components. One is light with a polarization parallel to the polarization of the light source (I_{∥}); and the other is light with a polarization perpendicular to the polarization of the light source (I_{⊥}). These two polarized components (that is, spectra) of the scattered light are collected separately with sensors near the light source. Then, I_{⊥} or a multiple of I_{⊥} is subtracted from I_{∥} to produce a fluorescence-free spectrum, Iₛₚ.

A benefit of this technique is that it allows Raman spectroscopy to use ultraviolet (UV) sources longer than 250 nm without fluorescence contamination. Typical UV Raman spectra utilizing light sources with wavelengths longer than 250 nm show considerable contamination from fluorescence when either an interrogated material, the surface it is on, or the container it is in has a high fluorescence quantum yield. Lasers with wavelengths below 250 nm are generally used because they avoid this problem, since the majority of the Raman spectrum occurs completely within a region outside the fluorescence interference. However, wavelengths less than 250 nm are often strongly absorbed and induce photodegradation leading to reduced Raman signals, though this is not always the case. In addition, wavelengths shorter than 250 nm are also harder to produce with currently available laser technology and only limited sources are available, many of which are not suitable for Raman because of long pulse lengths, broad line widths, use of toxic gases, low power output, poor efficiency, or wavelengths that are shorter than 230 nm that make it difficult to acquire optics for robust systems. Although the technique described herein can be used for laser wavelengths less than 250 nm, using longer-wavelength lasers with fluorescence removal from the Raman spectrum enables instrument designs that are more rugged; cheaper; and easier to design, produce, and maintain. Such instruments can also be more compact for a given power requirement as longer-wavelength lasers generally have higher wall-plug efficiencies.

This aspect of the invention also adds improved detection capabilities for a material of interest. The symmetric stretches in the Raman spectra of chemicals tend to be strong, but their intensity depends on the polarization of the light source relative to the polarization of the detector. For a given chemical, the higher the spectral intensity of a feature in a non-resonant Raman spectrum utilizing unpolarized sources, the more that feature's intensity will be subject to polarization effects. By observing which components in a Raman spectrum change as a function of the polarization of the detector relative to the light source, the origin of features can be ascertained. If this behavior for a material of interest is known at the time of obtaining an unknown Raman spectrum, then some features arising from the target material and the substrate or container of the material can be distinguished. Features not belonging to the material of interest may be ignored in subsequent detection algorithms for that material thereby removing algorithm confusion from spectral interference.

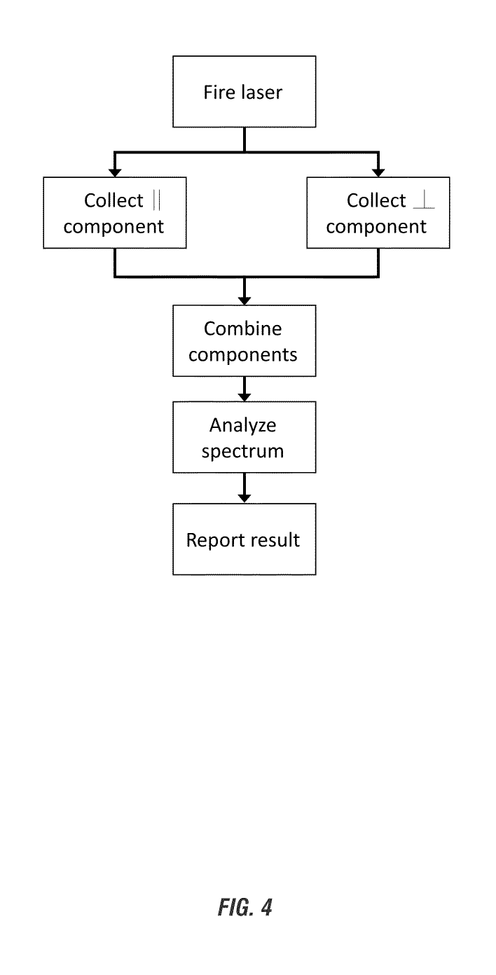

FIG. 4 is a high level flow chart of one example of the methodology according to the invention. As will be appreciated, the method can be carried out in a variety of ways with a variety of components. Examples will be discussed below.

The methodology of FIG. 4 can be implemented with a relatively inexpensive, robust, and portable laser, such as YAG-types which can generate laser energy in the UV ranges discussed herein. Collection of the parallel and perpendicular components of the returned light energy can be implemented with relatively inexpensive, robust and portable optical components. Combination of those components of light, analysis and reporting of results of the analysis can be implemented in relatively inexpensive, robust and portable devices such as spectrometers, iCCD cameras, and portable computers. All this promotes beneficial use for detection of chemicals non-destructively, quickly, and from standoff ranges.

Standoff ranges, typically of at least a meter, can be two or more meters, and even tens of meters. It is envisioned that the method of the invention can work (to at least some reasonable degree), up to distances of on the order of 100 meters. This would, of course, depend on a number of factors, including but not necessarily limited to, type and power of laser source, environmental conditions, the material under interrogation, the type of chemicals being monitored, the spectrograph and camera resolution, and throughput of the optic train. Therefore, it could work at even larger distances if conditions are right.

### Apparatus

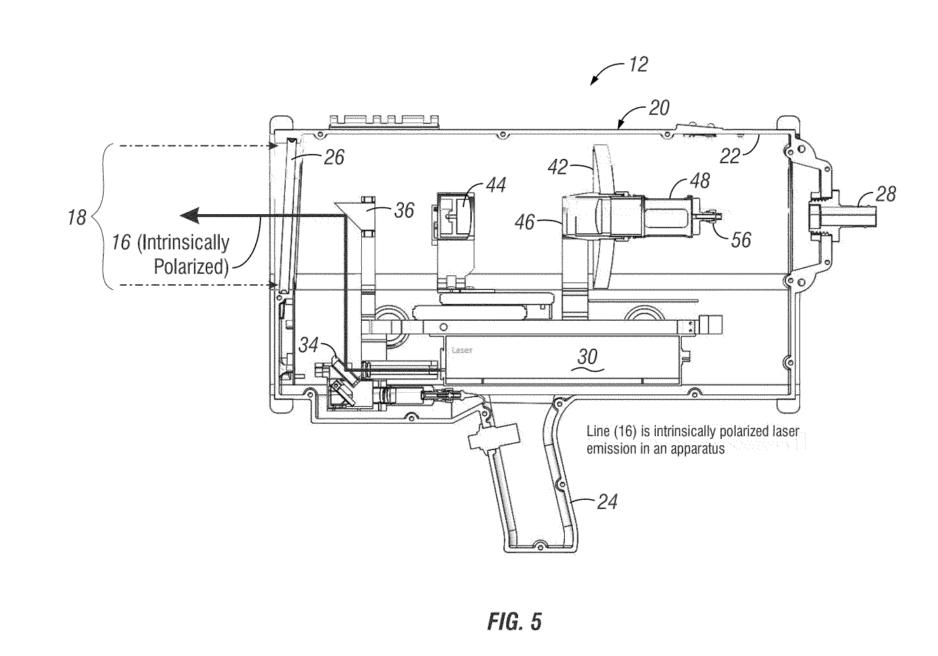

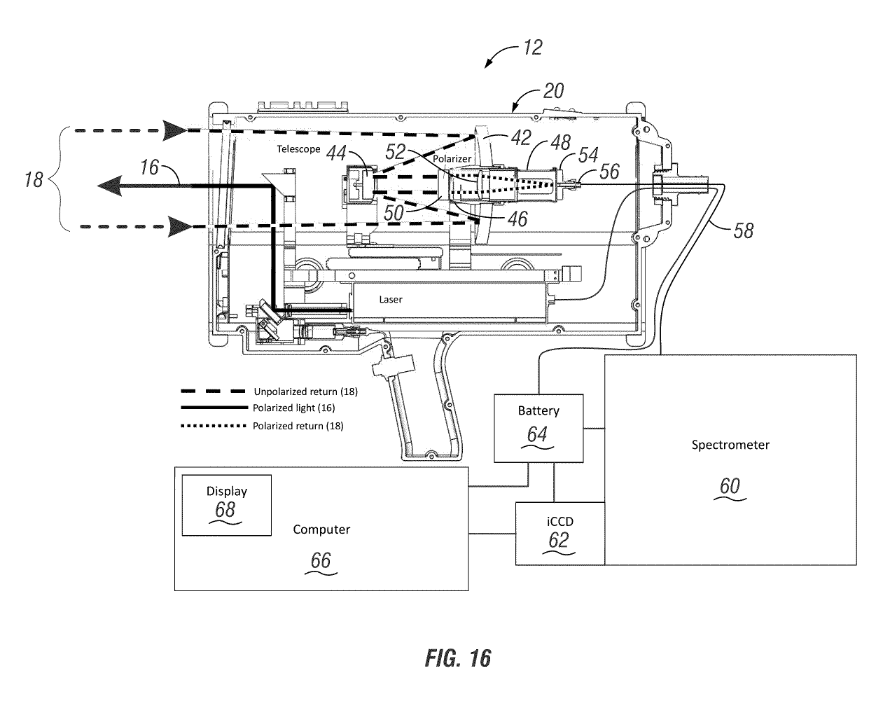

An apparatus for fluorescence removal in Raman spectra via collection and processing of polarized components is shown in FIG. 5. Reference can also be taken to the diagrammatic view in FIG. 3 and the overall system depiction of FIG. 16. The system (ref no. 10) includes the self-contained, hand-held apparatus 12 and ancillary and external components 14 to generate and emit and aim a laser beam 16 to a target (e.g. sample or material 19), and collect light 18 which can include back scattered and auto-emitted light (e.g. fluorescence) caused by the interaction of the laser on sample 19. These external components can include a spectrometer, imaging device, and computer, such as will be further described below.

In this embodiment, hand-held device 12 has a somewhat pistol-shaped overall body or housing 20 with a main internal chamber 22, a pistol-grip 24, a front end with light transmissive window 26, and a back end with a cable race or passage 28. Housing 20 can be made of a variety of materials, including but not limited to plastics, metals, composites, wood, and combinations of materials. It can be beneficial that they be durable, including for a wide range of outdoors environments, including rain, humidity, heat, sand, dust, dirt, and wind. By “hand-held” it is meant that apparatus 12 could be held and operated with a single hand of a typical person, such that size and weight do not preclude this. For example, the overall outside dimensions of apparatus 12 could be in the range of less than one foot between front and back ends, and much less than one foot in width and height. Weight could be less than 30 pounds. It is to be understood, however, that these would not necessarily be required.

The hand-held apparatus 12 includes a transmission source (e.g., laser 30), polarizing filters (e.g., filter 50), and a detector (e.g. spectrometer 60). The detector and laser are collocated to enable detection at standoff ranges. In this embodiment, the collocation is by configuring all components of system 10 to be portable, including by a single person.

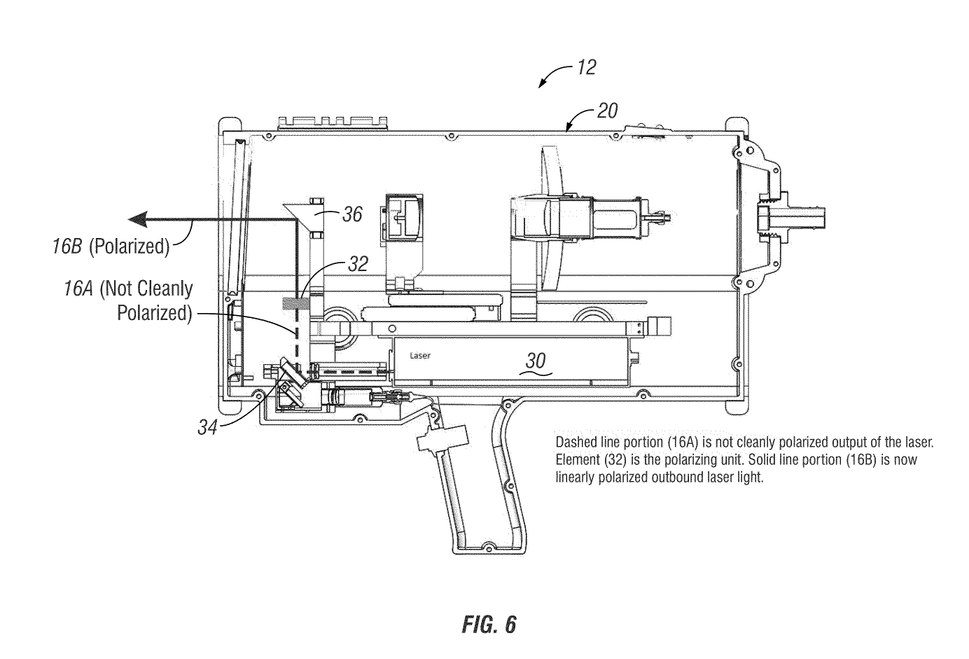

The laser source of the apparatus, along with the polarizing filters for the laser source are shown in FIG. 5. In this figure, the laser source is a solid-state UV laser 30 with a wavelength longer than 250 nm. The output 26 of this laser source 30 is linearly polarized either intrinsically (FIG. 5) or by using a movable filter 32 (FIG. 6). For the external filter embodiment of the source, the position of the filter 32 determines the plane of polarization of the laser output such that rotating the filter (relative to the axis of laser beam 16) changes the polarization. Once the plane of polarization of the laser source is set, the laser light is directed to a target (e.g. sample 19) that generates fluorescence and a Raman spectrum.

Intrinsically polarized laser sources are well-known and commercially available, including at the wavelengths of this embodiment. One commercially available example is Model QUV-355-150 from CrystaLaser of Reno, Nev. (USA). As indicated in FIG. 5, laser beam 16 could be directed from housing 20 by two 90 degree reflections at mirrors 34 and 36 to an output beam path through transmission window 26 that is basically parallel to the longitudinal axis of housing 20 between front and back ends. By techniques well-known to those skilled in the art, the mounting position of laser 30 in housing 20, the angle of mirrors 34 and 36, and the transmission window 26 can be calibrated with reference features (e.g. aiming sights) on the exterior of housing 20 to assist a user in sighting the laser beam 16 to a sample 19, including at standoff distances of a meter, several meters, several tens of meters, or more. A user can pick up hand-held apparatus 12 at hand grip 24, aim it, and actuate system 10 by, for example, a trigger or other manually-activated control at grip 24. If not intrinsically polarized in the fashion needed, polarizing filter 32 can be utilized. As diagrammatically illustrated in FIG. 6, filter 32 could be placed along the beam path of laser 30. In this example it is between mirrors 34 and 36. The portion directly out of laser 30 (see dashed line portion 16A), which is typically not cleanly polarized, is directed by mirror 34 through filter 32. The beam is thereafter (see solid line 16B) linearly polarized and continues to its target. Other positions are possible. In this embodiment it is moveable or adjustable in the sense it can be manipulated in situ between alternative orientations relative to beam 16. In this example, one position polarizes beam 16 in one orientation; a second position polarizes beam 16 in a different orientation. An example of such filter, rotatable approximately 90 degrees for the different polarization states is Model #89-552 from Edmund Optics, Barrington, N.J. (USA).

One example of allowing these two states is by simply adding a receiver along beam path 16 into which filter 32 can be mounted. The receiver would allow manual rotation between the two polarization states while holding filter 32 in beam path 16. Receiver and/or filter 32 could have either structural features or markings to help the user index the rotational position of filter 32 to the desired polarization state. Another example would be an electro-mechanical solution. Filter 32 could be held in beam 16 in a mount. Either the mount or filter 32 could be rotated by an electrically-powered actuator (actually a filter wheel) between states. One example would be a Nautilus Motorized Filter Wheel (Model OR-5526) from Orion, Watsonville, Calif. (USA). An external (or internal) switch or control could be manually activated by the user to select between polarization states. Alternatively, the system 10 could be calibrated to know or sense the states and automatically select between them. Other ways to affect polarization of beam 16 are possible; including but not limited to Brewster windows, optical surfaces, liquid-crystal polarizing filters, and fiber optic polarizing filters.

In this example, the fluorescence and Raman signals from the target 19 are radiated over 4π steradians. As mentioned, the Raman spectra have a dependence on the polarization of the laser light whereas the fluorescence contribution does not. A small portion of these signals make their way back to the instrument 12; the actual amount returned depends inversely on the square of the distance D_{SO} from the target to the instrument 12.

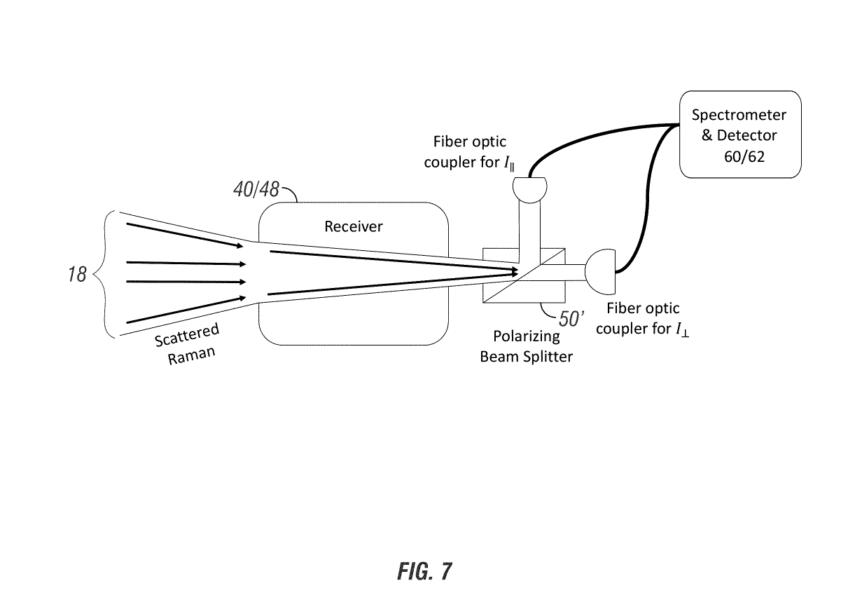

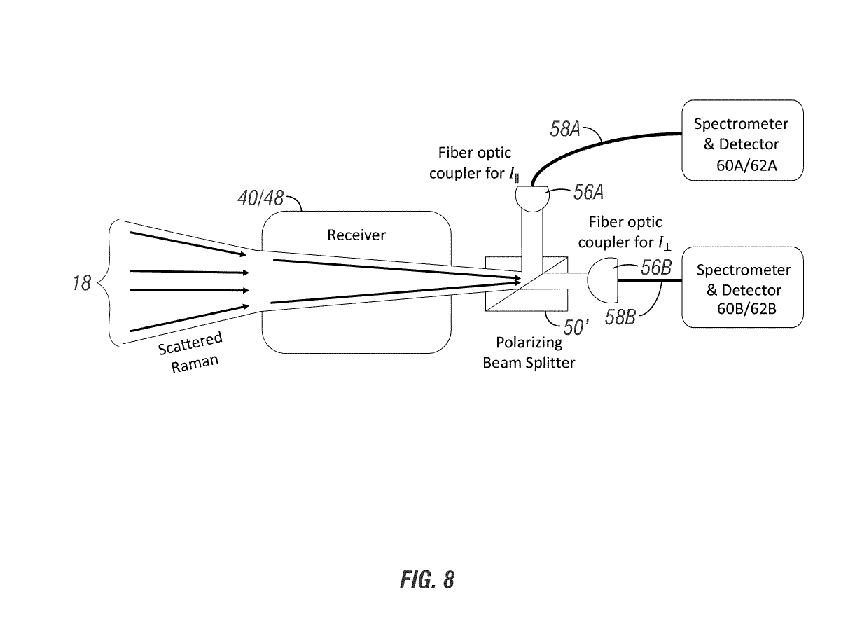

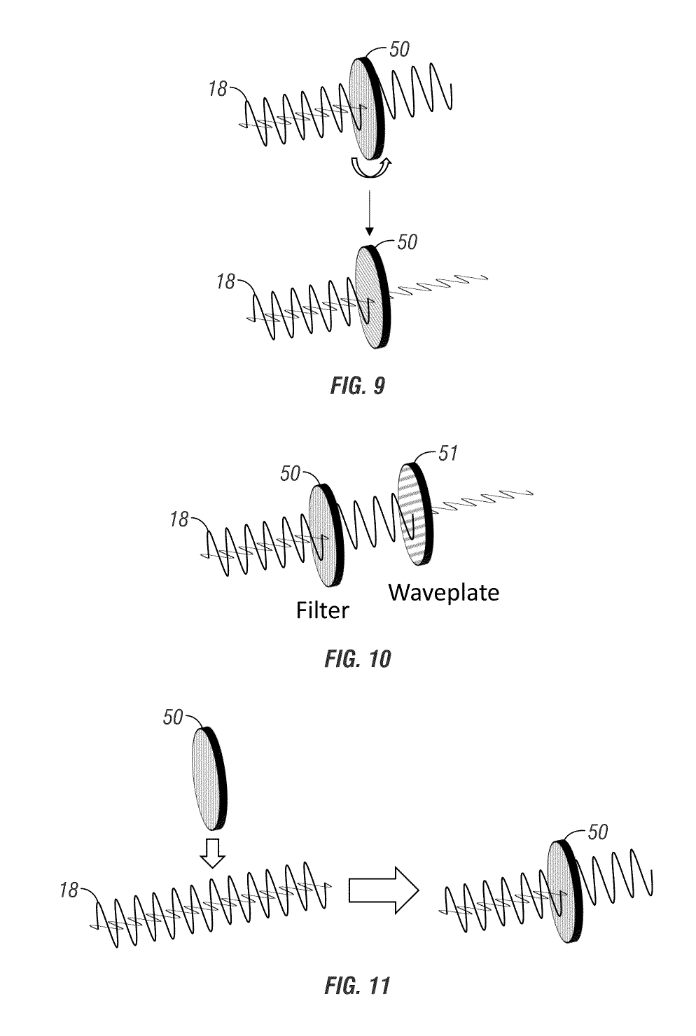

At the instrument 12, one or more optical configurations receive the fluorescence and Raman scattered light and pass it into one or more spectrometers 60 as in FIG. 7 and FIG. 8. See also FIG. 16. In the embodiments of FIGS. 7 and 8, the received light is split into parallel and perpendicular components without any adjustment of filters. Both polarizations are simultaneously collected. This can be beneficial instead of in front of the spectrometer(s) 60 having polarization filter(s) 50 where the filter(s) are rotated (or otherwise adjusted between polarization states) to receive the Raman light that is parallel or perpendicular to the laser plane of polarization (FIG. 9). Optionally, the polarization selection filter 50 can be fixed with a half-waveplate 51 rotated so as to alter the polarization of the incoming light, as in FIG. 10. In yet another embodiment of the technique, polarization filters set to a given polarization direction can simply be inserted into the apparatus as illustrated in FIG. 11. Regardless of the polarization selection method, the light is directed to a spectrometer or spectrometers that disperse the light to form a potentially fluorescence-contaminated Raman spectrum for a given polarization.

FIGS. 7 and 8 show possible embodiments where two outputs are made available. This would require two optical couplers 56A and 56B, each feeding an optical signal via its own fiber optic cable 58A and 58B to either a single spectrometer 60 (FIG. 7) or individual spectrometers 60A and 60B (FIG. 8).

As indicated in FIGS. 5-6, and diagrammatically at FIG. 3, collection of the light energy 18 for processing can utilize the same general optical axis as the emitted laser beam 16. Hand-held unit 12 is aimed at sample 19. Because of the physics of light, the back-scattering and fluorescence generate at sample 19 upon irradiation with laser beam 16 and, at the speed of light, travel basically omni-directionally from sample 19, including a portion back along the optical axis of laser 16 and through window 26 of hand-held unit 12. The aperture (height and width) of window 26 allows into cavity 22 any such light. In this embodiment, that collected light is further collected and focused by what is essentially a catadioptric telescope 40, using mirrors and lenses to form an image of the light collected through window 26. Telescope 40 includes large converging mirror 42, which captures and converging reflects incident light to secondary and smaller mirror 44. See also FIG. 16. Mirror 44 basically collimates the light to a focusing chamber 48. Focusing chamber 48 includes a mount 46 at its entrance opening. Mount 46 is adapted to receive polarizing filter 50, or a receiver or holder of filter 50, in a manner that allows the rotational (or other adjustment) between polarizing states. Other light collecting and focusing techniques are possible.

As indicated, one configuration for such a mount is simply a receiver or holder that allows a user to manually rotate filter 50 relative the optical axis of the collected light between polarization states (perpendicular or parallel). Alternatively, there could be an electro-mechanical or other technique, such as discussed regarding filter 32 earlier. In any case, this allows presentation of different polarization states, and thus different polarizations, to the collected light.

Focusing chamber 48 can include a focusing lens 52 (and/or other optical components) to focus the light to an optical coupler 56 mounted at back end 54 of focusing chamber 48, to optical cable 58 (e.g. fiber optic).

Optical cable 58 extends through rear port 28 of housing 20 to external components 14. Spectrometer(s) 60 (there could be one or more) receive the collected light from hand-held unit 12 through optical cable 58 and, through conventional techniques well known in the art, produce spectra from such light.

As will be appreciated by those skilled in the art, the specific components and relationship of components to generate the polarized laser beam 16 and collect and focus return light can vary according to need and desire. In this embodiment, they are packaged in a portable, substantially self-contained housing for convenient use in the field. As also can be appreciated, the laser source and the other components can be relatively economical and easy to assemble into the housing, at least as compared with such laser sources as excimer lasers. Laser sources of the type discussed with respect to this embodiment (e.g. YAG) are commercially available, relatively small in size and weight, economical, and robust, and can generate the needed wavelength laser light for system 10. One example of such a laser source is Model QUV-355-150 commercially available from CrystaLaser of Reno, Nev. (USA).

As will be further appreciated, the ability to use either an intrinsically polarized laser source or add a polarizing component external of the source, provides flexibility. Still further, the technique of being able to adjust polarization of the beam such as with a polarizing component external of the source allows further flexibility.

Housing 20 can have appropriate doors or access to internal components, such as if manual adjustment of either polarizing element 32 or 50 is allowed, or calibration, adjustment, replacement, or maintenance is needed or desired for internal contents. FIGS. 5, 6, and 16 are shown with essentially the entire left side wall of housing 20 removed for clarity. But it can be appreciated that dedicated and smaller lids, doors, panels, or other access techniques are possible.

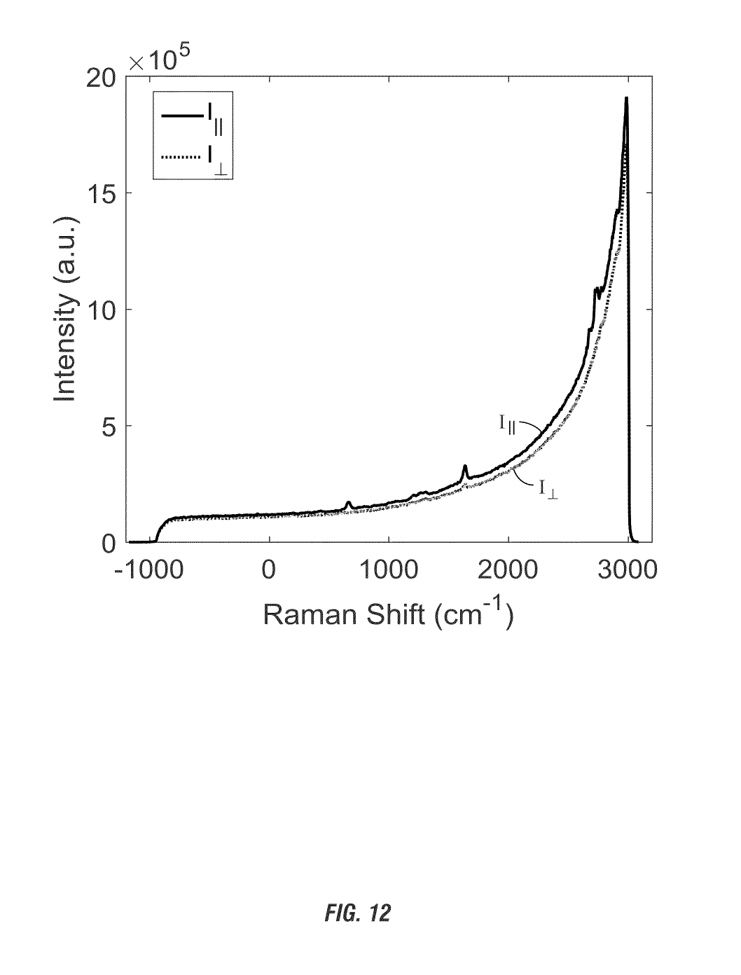

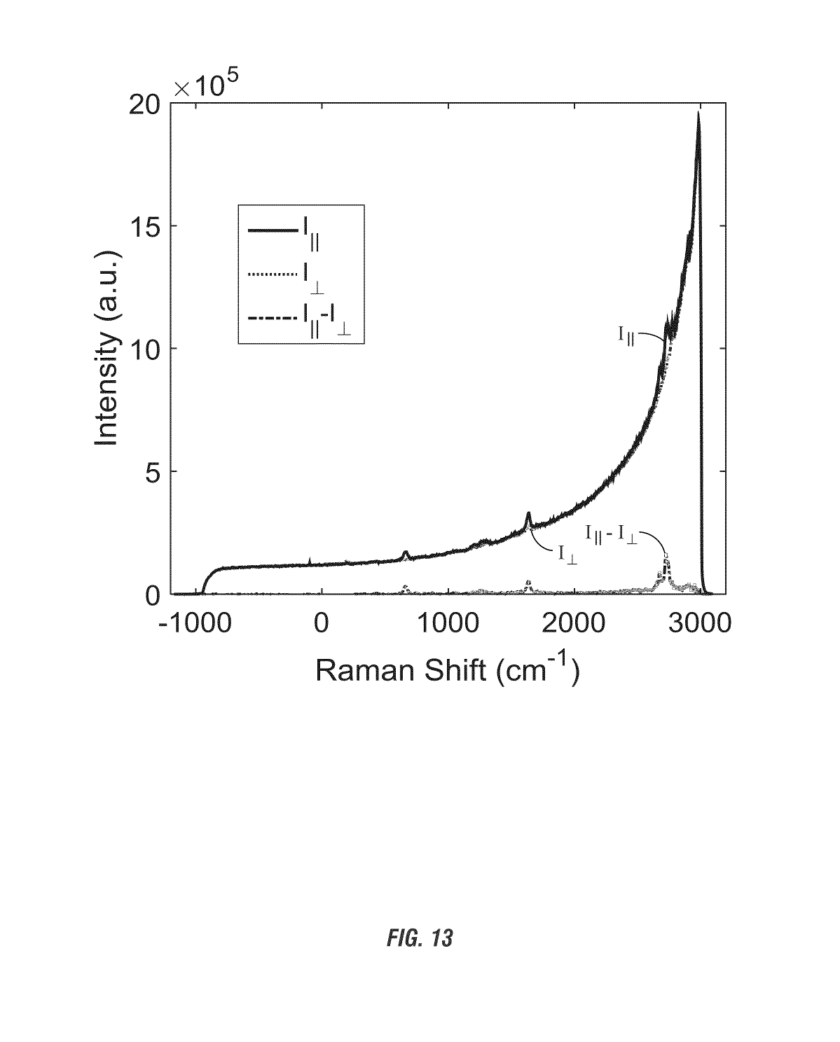

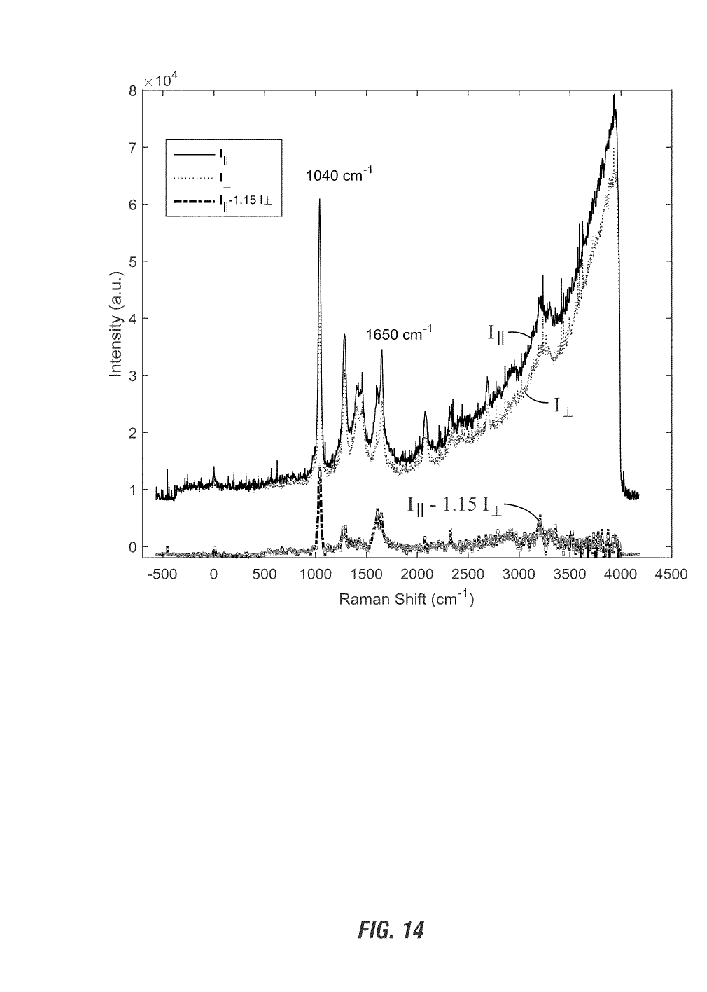

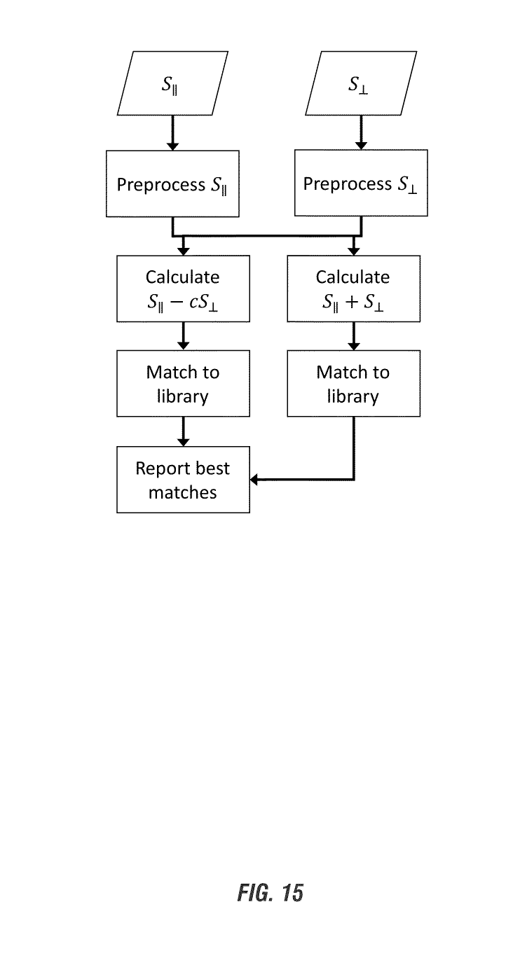

A detector (e.g. image intensified charge coupled device (iCCD) camera 62) attached to the spectrometer 60 records the fluorescence-contaminated Raman spectrum and sends it to a computer or digital processer (e.g. computer 68) where signal processing and decisions about the Raman spectrum occurs. Each component of the Raman spectrum is read separately and stored in memory on a computer 68. The components are then possibly preprocessed and run through a spectral recombination algorithm that directly subtracts I_{⊥} from I_{∥} or subtracts a scaled version of I_{⊥} from I_{∥} to form a new spectrum that contains the most symmetric normal mode features as in FIG. 14. An example of scaled and unscaled spectra utilizing DMMP, as well as the subtraction result can be seen in FIG. 12 and FIG. 13. A similar technique for ammonium nitrate mixed with fuel oil (ANFO) is shown in FIG. 14. The new unknown, subtracted spectrum—after potentially more preprocessing—is then compared to a set of Raman spectra of known chemicals; these known spectra were previously collected with the instrument and were stored in a library. Chemicals with library spectra with a strong match as determined by the decision algorithm are deemed to be present in the target material. The decision process may also use the sum of I_{⊥} and I_{∥} (e.g. S_{⊥} and S_{∥}) if it is determined that no fluorescence is present in the returned spectrum or if this spectrum and the difference spectrum are both needed to make an accurate decision. A flowchart of the signal processing and the decision processes appears in FIG. 15.

As can be appreciated by those skilled in the art, the software programming for the above-discussed processing can vary according to need or desire. Likewise can the spectrometer, detector, and computer; commercially available examples of which are a Shamrock 303i from Andor Technology Ltd of Belfast BT12 7AL, UK; an Andor iStar DH334T-18F-03 intensified CCD array from Andor Technology Ltd of Belfast BT12 7AL, UK; and a Model Gb-bxi3h-4010 from Giga-Byte Technology Co., LTD of New Taipei City 231, Taiwan; respectively. Optical components can be selected from commercially available sources also. The spectrometer can have, in one example, 2400 grooves per mm. The computer can have a PC104 form factor and be a single board computer.

Finally, an example of an apparatus and overall system 10 capable of this technique according to aspects of the invention is diagrammed in FIG. 16. This system 10 includes a handheld laser source and receiver 12, such as above described. The laser is intrinsically polarized. Scattered light is returned back to the instrument 12 and collected with a telescope (which could be built into housing 20 or could simply be external and separately hand-held. Afterwards, the collected light is then passed through an adjustable polarizer 50 and into an optical fiber 58, where it passes through to a spectrometer 60 that disperses the light onto a detector (an iCCD 62 in this case) that is read out by a computer 66 to obtain the polarized Raman spectrum. Next, spectra with the polarizer 50 rotated 90° are collected before both polarization spectra are preprocessed and combined as in FIG. 15. The computer 66 then performs an analysis to determine the presence of a chemical. An example of that processing (e.g. comparing the extracted Raman spectra to a library of known reference spectra available to computer 66), has been discussed earlier.

In one embodiment, the external components of system 10 (external of hand-held device 12) can be portable by one person as follows. A carrier, for example a backpack, could be configured to hold spectrometer 60 and iCCD camera 62. Computer 66 could be included. So to could a portable power source (e.g. battery 64). Appropriate connections (wired or unwired) would be configured as needed. Thus, overall system 10 could be efficiently and effectively carried and operated by a single person.

As can be appreciated by those skilled in the art, computer 66 could take many forms and embodiments. One example would be the Model Gb-bxi3h-4010 from Giga-Byte Technology Co., LTD of New Taipei City 231, Taiwan (under 3 lbs. and 1.69 in×4.24 in×4.5 in). Other portable, lunchbox, or luggable computers are possible. It may be possible to also use small laptops, tablets, notebooks, or even appropriately powerful smart phones as the mobile computing device 66. The computer can include a display 68.

The battery 64 can be selected to provide portable electrical power for one or more of computer(s) 60, detector 62, spectrometer(s) 60, and laser source 30. It could also supply power to any electrical or electromechanical actuator(s) such as might be used to rotate or adjust polarizing filter(s) 32 or 50, or other components. A commercially available example is Model PMD-CP12266 from PowerStream Technology of Orem, Utah (USA).

It can therefore be seen that the embodiments meet at least one aspect, feature, advantage, or object of the invention.

In one form, a hand-held instrument includes a polarized UV laser source to generate an interrogating laser beam to standoff distances. It can utilize an intrinsically polarized laser or can include another polarizing element in the hand-held instrument and external of the laser source which can be set to one polarization state or optionally adjusted between at least two different polarization states. The hand-held device allows “point and shoot” of the laser beam to the target (e.g. sample under interrogation). The laser beam is polarized to a pre-known polarization state.

A collector of return light from the interrogation is also at or built into the hand-held device. In one form it is basically a telescope which collects and focuses light in its field of view (which includes any light from the interrogation in that field of view). The optical manipulation of that gathered light is such that it can be effectively communicated to a spectrometer. In one form this is by conventional use of an optical coupler of the focused light into a fiber optic cable operatively connected to the spectrometer. Prior to communication to the fiber optic, the return light is intentionally polarized. A polarizer element is interposed in the optical path of the return light and can be adjusted between different polarization states. In one form this can be a simple rotation of a polarizer 90 degrees. Spectra of the return light are produced in a portable spectrometer operatively connected to the hand-held instrument, each polarized in a different polarization state produced by the adjustment of the polarizer element in the hand-held device. Those spectra are quantified and compared in a portable computer. The comparison can be used to remove fluorescence and better distinguish Raman information to more accurately detect constituent chemicals in the return light. The hand-held instrument includes structure to allow quick and easy adjustment of the polarizing element between the two polarization states. Utilizing UV laser sources and the adjustable polarization states allows a portable, outdoors field useable, relatively economical system for standoff distances, including meters to tens of meters.

### Options and Alternatives

Numerous modifications may be made to the apparatus or the invention, particularly to the detection algorithm, without departing from its scope as defined in appended claims. This is likewise for the apparatus components.

The foregoing descriptions are examples only of the forms and embodiments the invention may take. Variations obvious to those skilled in the art will be included within the invention. Several examples of variations have been discussed above.

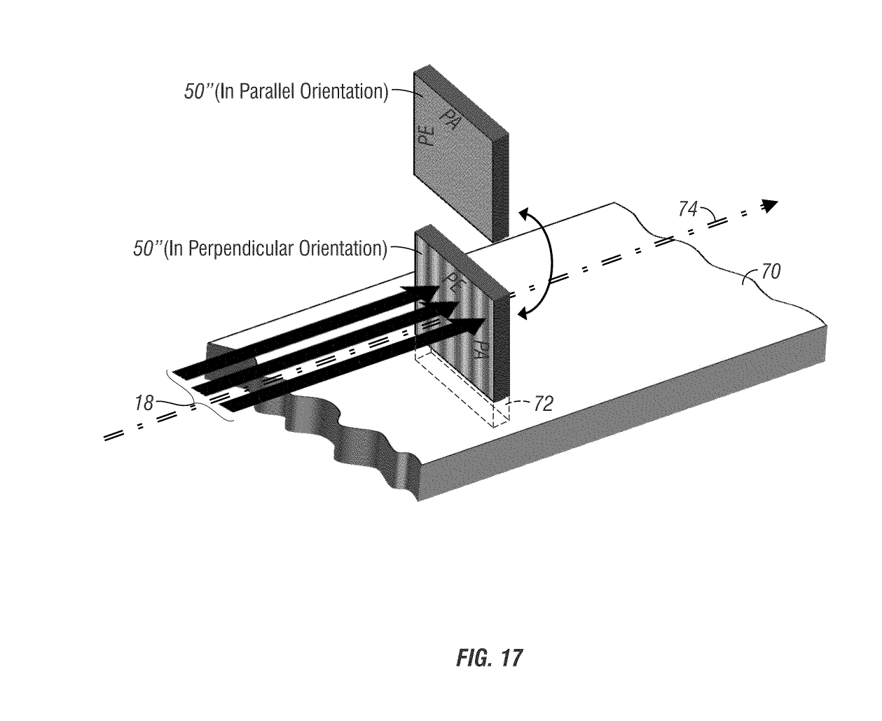

One example is how the return light polarizer can be adjusted between polarization states. As mentioned previously, it could be simply rotating the element. This can be accomplished in a variety of ways. Just one example is illustrated at FIG. 17. A plate 70 could be mounted to the front end 46 of collector 40 inside housing 20 (see FIG. 5) below the center of optical path 74 that goes to optical coupler 56. A simple rectangular slot 72 in the top surface of plate 70 can be sized for complementary fit of a square polarizer element 50″. Element 50″ has a first polarization state at one rotational orientation (e.g. marked “PA” on polarizer 50″ (for parallel) in FIG. 17) and a second polarization state at 90 degrees rotation to the first (e.g. marked “PE” on polarizer 50″ (for perpendicular)). The user simply places polarizer 50″ in slot 72 with the desired marking (PA) or (PE) in the top or up position to select the polarization state. Polarizer 50″ will be in a consistent and repeatable orientation relative to the optical path 74 (and the return light energy 18) for either state. Instead of a slot 72 in surface 70, the bottom squared edge of filter 50″ could be positioned on flat surface of plate 70 and held in position by other structure (e.g. clip, clamp, etc.). Other techniques are possible including, but not limited to, using a Brewster window to split the incoming light into I_{∥} and I_{⊥} and a series of mirrors to pick the polarization of interest, changing the polarization of a transmissive liquid-crystal polarization filter, or by inserting elements such as polarizing fiber optics in place of the square polarizer mentioned here. Other examples are as follows:

The Sample or Material Under Interrogation

As indicated above, the analyzed material can take different forms. Non-limiting examples are solid, liquid, gas, or mixture of states; a mixture of chemicals in various states; an explosive; or a hazardous substance or a Raman interferent for a hazardous substance. The material can be isolated, on a surface; or in a container. Beneficial results can be best for liquids or thin layers.

The Hand-Held Housing and Other System Components

The system can be configured with a ruggedized laser source, housing, processor, power supply, and control system for indoor or out of doors use for chemical constituents including but not limited to toxic materials and explosives. This can include the hand-held housing and its contents, as well as the components external to it, such as spectrometer(s), camera(s), and computer.

The Computer

The computer can include a data storage component and display component to store and display information, including the determination made regarding the material under interrogation. A smartphone is considered one example of this type of computer.

The software can comprise a signal processing algorithm whereby polarization is used to discriminate materials against a spectral background or against other materials of interest.

The Laser Source

The laser source can take different forms. Non-limiting examples are a UV laser, a UV laser with a wavelength between 220 and 400 nm: a solid-state UV laser; an excimer laser. Non-limiting examples of laser operation include pulsed or continuous wave (cw) or pseudo-cw.

Further non-limiting examples of the laser are a frequency-tripled or quadrupled Nd³⁺:YAG laser; a frequency-tripled or quadrupled Yb³⁺:YAG laser; a frequency-tripled or quadrupled Nd³⁺:YLF laser; a Tm³⁺:YALO laser operating at the 8^{th} harmonic frequency; or any similar solid-state laser, such as a Ti³⁺:Sapphire, VCSEL, or VECSEL laser operating at a harmonic frequency in the UV region.

In embodiments discussed in earlier sections, the laser source was UV and above 250 nm wavelengths. Further non-limiting examples are:
a. wavelength of 220-250 nm;
b. wavelength of 250-270 nm;
c. wavelength of 270-320 nm
d. wavelength of 320-360 nm; or
e. wavelength of 360-400 nm.

Polarization of the Laser

The laser source can comprise an intrinsically linearly polarized ultraviolet (UV) laser. It is envisioned that fluorescence reduction can be achieved at a factor of 5 or greater for materials where fluorescence interferes with the Raman spectrum.

The laser source can comprise a UV laser and a polarization filter external to the laser cavity and is also envisioned to achieve fluorescence reduction by a factor of 5 or greater for materials where fluorescence interferes with the Raman spectrum.

Selecting the polarization of a laser source can vary. Non-limiting examples are the laser polarization is switched using a polarization filter which is rotated to different orientations; the laser polarization is switched using a fixed polarization filter and a waveplate rotated to different orientations; the laser polarization is switched by inserting one or multiplicity of polarization selective optics.

Polarizing the Received/Returned Light from the Interrogation

Receiving, or collecting and focusing, the return light from the interrogation can be done in different ways. Non-limiting examples are the receiver is a telescope; or the receiver is a collection of lenses, mirrors, and related focusing optics. Non-limiting examples of Raman scattering include Raman scattering which originates from the material, surface, or container, from atmosphere, or from some combination of them; some constituent of the material, the atmosphere, the surface, or the container fluoresces in the same region as the Raman scattering

Selecting between polarization states for polarizing the return light from the interrogation can vary. Non-limiting examples are, the receiver polarization is switched using a polarization filter which is rotated to different orientations; the receiver polarization is switched using a fixed polarization filter and a waveplate rotated to different orientations; the receiver polarization is switched by inserting one or multiplicity of polarization selective optics; or the received light is split into two signals using a beam splitter before passing each through a polarization filter.

Processing of the spectra from the spectrometer can vary. Non-limiting examples include the case where the polarized Raman spectrum that is perpendicular to the polarization of the laser source is directly subtracted from the polarized Raman spectrum that is parallel to the polarization of the laser source; the polarized Raman spectrum that is perpendicular to the polarization of the laser source is scaled before being subtracted from the polarized Raman spectrum that is parallel to the polarization of the laser source; or the polarized Raman spectra are preprocessed before performing spectral combination.

Still further additional features, aspects, options, and alternatives regarding handling of the collected radiation from the interrogation can include the following non-limiting examples:
a. The ratio of the parallel polarized Raman spectrum to the perpendicular polarized Raman spectrum can be in the range of 2:1 to 100:1.
b. The two received polarizations can be selected so that one is substantially the same polarization as the laser and the other is substantially perpendicular to the polarization the laser.
c. The two received polarizations can be selected so that one is substantially the same polarization as the laser and the other is substantially perpendicular to the polarization the laser.
d. The receiver polarization can be switched using a polarization filter which is rotated to different orientations.
e. The receiver polarization can be switched using a fixed polarization filter and a waveplate rotated to different orientations.
f. The receiver polarization can be switched by inserting one or another of a multiplicity of polarization selective optics.
g. The two received polarizations can be simultaneously measured by monitoring both transmitted and reflected light from a polarizing element.
h. The receiver polarization can be fixed and the laser polarization is changed.
i. The collected light can be split based on polarization into two collectors.
j. The collectors and spectrometer(s) can be located at less than 10° of the angle made by the source, interrogated material, and receiver or spectrometer.
k. The quantified perpendicular scattering can be denominated spectrum component I_{⊥}, the quantified parallel scattering can be denominated I_{∥}, and a combination of I_{⊥} and I_{∥} can be denominated Iₛₚ, and in which the combination of polarized spectra Iₛₚ can be calculated by subtracting the perpendicular spectrum I_{⊥} from the parallel spectrum I_{∥} where

Iₛₚ=I_{∥}−I_{⊥}
l. The quantified perpendicular scattering can be denominated spectrum component I_{⊥}, the quantified parallel scattering can be denominated I_{∥}, and a combination of I_{⊥} and I_{∥} can be denominated Iₛₚ, and in which the combination spectrum Iₛₚ can be calculated by multiplying the perpendicular spectrum by a scaling factor c and then subtracting from the parallel spectrum.

Iₛₚ=I_{∥}−cI_{⊥}
m. The components I_{∥} or I_{⊥} can be first preprocessed before creating the combination spectrum, Iₛₚ.
n. The increase in the ratio of Raman to fluorescence signal in the signal, wherein the signal is denominated Iₛₚ, can be on the order of at least approximately 5 or more times over conventional unpolarized UV Raman spectroscopy.
o. The laser source cam comprise a polarized UV laser having a wavelength and flipping between parallel and perpendicular at a rate at a receiver.

As can be appreciated, the references to I_{∥} or I_{⊥} could be changed to S_{∥} or S_{⊥}.

Other Optics

As will be appreciated by those skilled in the art, other optical components may be used in the system. Non-limiting examples are as follows. A Rayleigh scattering rejection filter aka as a “laser line filter” could be used to reject Rayleigh scattering that shows up around a shift of 0 cm⁻¹. The actual width of this scattering depends on the bandpass of the filter, and the scattering can spread out over a few hundred wavenumbers (cm⁻¹), so this filter can be beneficial with aspects of the invention. A fluorescence rejection filter could be used if needed. It could be included to reject stray light from fluorescence outside the detector region that hits the detector. The detector senses any light that falls on it regardless of wavelength (or Raman shift). Normally, only Raman scattering and the fluorescence in the wavelength region set by the spectrometer hits the detector, but sometimes stray light (from fluorescence or just ambient stuff) makes it into the spectrometer and reflects inside it until the light strikes the detector, which necessitates this optic.

### REFERENCES

[1] S. T. McCain, R. M. Willett, D. J. Brady, Multi-excitation Raman spectroscopy technique for fluorescence rejection., Opt. Express. 16 (2008) 10975-91.
[2] A. C. De Luca, M. Mazilu, A. Riches, C. S. Herrington, K. Dholakia, Online fluorescence suppression in modulated Raman spectroscopy., Anal. Chem. 82 (2010) 738-45.
[3] E. C. Le Ru, L. C. Schroeter, P. G. Etchegoin, Direct measurement of resonance Raman spectra and cross sections by a polarization difference technique., Anal. Chem. 84 (2012) 5074-9.
[4] S. A. Asher, Ultraviolet Raman Spectroscopy, in: J. M. Chalmers, P. R. Griffiths (Eds.), Handb. Vib. Spectrosc., John Wiley & Sons, Ltd, Chichester, U K, 2006.
[5] L. Nagli, M. Gaft, Y. Fleger, M. Rosenbluh, Absolute Raman cross-sections of some explosives: Trend to UV, Opt. Mater. (Amst). 30 (2008) 1747-1754.
[6] L. Wang, D. Tuschel, S. a. Asher, 229 nm UV Photochemical Degradation of Energetic Molecules, in: A. W. Fountain III, P. J. Gardner (Eds.), Proc. SPIE, 2011: p. 80181B-80181B-6.
[7] M. A. da Silva Martins, D. G. Ribeiro, E. A. Pereira Dos Santos, A. A. Martin, A. Fontes, H. da Silva Martinho, Shifted-excitation Raman difference spectroscopy for in vitro and in vivo biological samples analysis., Biomed. Opt. Express. 1 (2010) 617-626.
[8] Z. Zhang, S. Chen, Y. Liang, Z. Liu, Q. Zhang, L.-X. Ding, et al., An intelligent background-correction algorithm for highly fluorescent samples in Raman spectroscopy, J. Raman Spectrosc. 41 (2009) 659-669.
[9] L. Nagli, M. Gaft, Y. Fleger, M. Rosenbluh, Absolute Raman cross-sections of some explosives: Trend to UV, Opt. Mater. (Amst). 30 (2008) 1747-1754.
[10] D. A. Long, Intensities in Raman Spectra. I. A Bond Polarizability Theory, Proc. R. Soc. A Math. Phys. Eng. Sci. 217 (1953) 203-221.
[11] J. R. Lakowicz, ed., Principles of Fluorescence Spectroscopy, Springer US, Boston, Mass., 2006.
[12] C. D. Allemand, Depolarization Ratio Measurements in Raman Spectrometry, Appl. Spectrosc. 24 (1970) 348-353.
[13] S. Christesen, Improved Raman sensitivity using polarization analysis, in: A. W. Fountain III, P. J. Gardner (Eds.), Proc. SPIE, 2010: p. 76651B-76651B-7.
[14] J. Egermann, T. Seeger, A. Leipertz, Application of 266-nm and 355-nm Nd:YAG Laser Radiation for the Investigation of Fuel-Rich Sooting Hydrocarbon Flames by Raman Scattering, Appl. Opt. 43 (2004) 5564.
[15] E. C. Le Ru and P. G. Etchegoin, Plasmon-mediated Polarization Anomalies in Surface Enhanced Raman Scattering (SERS), arXiv:physics/0504182v7 [physics. chem-ph] 26 Apr. 2005.
[16] P. Kowalska, J. R. Cheeseman, K. Razmkhah, F. Green, L. A. Nafie, and A. Roger, Experimental and Theoretical Polarized Raman Linear Difference Spectroscopy of Small Molecules with a New Alignment Method Using Stretched Polyethylene Film, Anal. Chem. 2012, 84, 1394-1401.

What is claimed is:

1. A method of standoff detection of a material on a surface or in a container comprising:
a. interrogating a suspected material with a laser source having a linear polarization from a standoff distance;
b. collecting scattering from the combined material, surface, and/or container at polarizations parallel and perpendicular to the polarization of the source with a spectrometer located proximally to the light source;
c. quantifying the collected scattering;
d. calculating a combined spectrum of the collected quantified parallel polarization scattering from the quantified perpendicular polarization scattering; and
e. evaluating the combination result for a signal indication of an explosive.

2. The method of claim 1 where the material is a chemical or a mixture of chemicals.

3. The method of claim 1 where the material is a solid, liquid, gas, or mixture of states.

4. The method of claim 1 where the material is a hazardous substance or a Raman interferent for a hazardous substance.

5. The method of claim 1 where the scattering includes Raman scattering which originates from the material, surface, or container, from atmosphere, or from some combination of them.

6. The method of claim 5 where some constituent of the material, the atmosphere, the surface, or the container fluoresces in the same region as the Raman scattering.

7. The method of claim 5 wherein the ratio of the parallel polarized Raman spectrum to the perpendicular polarized Raman spectrum is in the range of 2:1 to 100:1.

8. The method of claim 1 wherein the standoff distance is at or beyond a meter.

9. The method of claim 1 wherein the laser source is compact, rugged, economical, and simple to maintain.

10. The method of claim 1 wherein the laser source is an excimer laser.

11. The method of claim 1 wherein the laser source comprises a solid state laser.

12. The method of claim 1 wherein the laser source is pulsed, continuous-wave, or pseudo continuous-wave.

13. The method of claim 11 wherein the laser comprises a frequency-tripled or quadrupled Nd³⁺:YAG laser; a frequency-tripled or quadrupled Yb³⁺:YAG laser; a frequency-tripled or quadrupled Nd³⁺:YLF laser; a Tm³⁺:YALO laser operating at the 8^{th} harmonic frequency; or any similar solid-state laser, such as a Ti³⁺:Sapphire, VCSEL, or VECSEL laser operating at a harmonic frequency in the UV region.

14. The method of claim 11 wherein the laser has at least one of:
a. wavelength of 220-250 nm;
b. wavelength of 250-270 nm;
c. wavelength of 270-320 nm;
d. wavelength of 320-360 nm;
e. wavelength of 360-400 nm.

15. The method of claim 1 wherein the laser source comprises an intrinsically linearly polarized ultraviolet (UV) laser and claims fluorescence reduction is a factor of 5 or greater for materials where fluorescence interferes with the Raman spectrum.

16. The method of claim 1 wherein the laser source comprises a UV laser and a polarization filter external to the laser cavity and wherein the fluorescence reduction is a factor of 5 or greater for materials where fluorescence interferes with the Raman spectrum.

17. The method of claim 1 where the collector is a telescope.

18. The method of claim 1 where the collector is any single element or combination of lenses, mirrors, or other focusing optics.

19. The method of claim 1 where the collector collects scattered Raman and fluorescence light generated by the laser source.

20. The method of claim 19 wherein the two received polarizations are selected so that one is substantially the same polarization as the laser and the other is substantially perpendicular to the polarization the laser.

21. The method of claim 19 in which the receiver polarization is switched using a polarization filter which is rotated to different orientations.

22. The method of claim 19 in which the receiver polarization is switched using a fixed polarization filter and a waveplate rotated to different orientations.

23. The method of claim 19 in which the receiver polarization is switched by inserting one or another of a multiplicity of polarization selective optics.

24. The method of claim 19 in which the two received polarizations are simultaneously measured by monitoring both transmitted and reflected light from a polarizing element.

25. The method of claim 19 in which the laser polarization is changed.

26. The method of claim 1 in which the collected light is split based on polarization into two collectors.

27. The method of claim 26 in which the collectors and spectrometer(s) are located at less than 10° of the angle made by the source, interrogated material, and receiver or spectrometer.

28. The method of claim 1 wherein the quantified perpendicular scattering is denominated spectrum component I_{⊥}, the quantified parallel scattering is denominated I_{∥}, and a combination of I_{⊥} and I_{∥} is denominated Iₛₚ, and in which the combination of polarized spectra Iₛₚ is calculated by subtracting the perpendicular spectrum I_{⊥} from the parallel spectrum I_{∥} where
Iₛₚ=I_{∥}−I_{⊥}

29. The method of claim 1 wherein the quantified perpendicular scattering is denominated spectrum component I₁, the quantified parallel scattering is denominated I_{∥}, and a combination of I_{⊥} and I_{∥} is denominated Iₛₚ, and in which the combination spectrum Iₛₚ is calculated by multiplying the perpendicular spectrum by a scaling factor c and then subtracting from the parallel spectrum.
Iₛₚ=I_{∥}−cI_{⊥}

30. The method of claim 28 in which the components I_{∥} or I_{⊥} are first preprocessed before creating the combination spectrum, Iₛₚ.

31. The method of claim 29 in which the components I_{∥} or I_{⊥} are first preprocessed before creating the combination spectrum, Iₛₚ.

32. An apparatus for non-destructively interrogating a substance from a distance for at least one chemical constituent comprising:
a. a laser source generating directed, polarized laser energy in the UV wavelength range;
b. a receiver having components for collecting Raman scattering of the source parallel and perpendicular to the source polarization and generating signals correlated to the parallel and perpendicular collected polarizations;
c. a processor having an input for the signals;
d. software for signal acquisition, data processing and material detection to
i. combine the collected parallel and perpendicular polarizations;
ii. generate a signal representative of the combination;
iii. determine if the content of the combination is indicative of a chemical constituent.

33. The apparatus of claim 32 wherein the laser source comprises a solid state or excimer laser having relatively small form factor.

34. The apparatus of claim 33 in combination with a housing having a relatively small form factor.

35. The apparatus of claim 34 wherein the laser has at least one of:
a. wavelength of 220-250 nm;
b. wavelength of 250-270 nm;
c. wavelength of 270-320 nm;
d. wavelength of 320-360 nm;
e. wavelength of 360-400 nm.

36. The apparatus of claim 32 wherein the laser source comprises a polarized UV laser having a wavelength and flipping between parallel and perpendicular at a rate at a receiver.

37. The apparatus of claim 32 wherein the increase in the ratio of Raman to fluorescence signal in the signal, wherein the signal is denominated Iₛₚ, is on the order of at least approximately 10 times over conventional unpolarized UV Raman spectroscopy.

38. The apparatus of claim 32 in combination with a ruggedized laser source, housing, processor, power supply, and control system for indoor or out of doors use for chemical constituents including but not limited to toxic materials and explosives.

39. The apparatus of claim 32 in combination with a data storage component and display component to store and display the determination.

40. The apparatus of claim 32 wherein the software comprises a signal processing algorithm whereby polarization is used to discriminate materials against a spectral background or against other materials of interest.

41. A system for standoff distance interrogation of an unknown sample comprising:
(a) a hand-held instrument including:
(i) a polarized UV laser source to generate an interrogating laser beam to standoff distances, and
(ii) a collector of return light from the interrogation, and a polarizer of the return light that can be adjusted between different polarization states;
(b) a portable spectrometer operatively connected to the hand-held instrument and adapted to receive spectra of the return light, each polarized in a different polarization state; and
(c) a portable computer operatively connected to the spectrometer and adapted to quantify and compare the spectra, the comparison used to remove fluorescence and better distinguish Raman information to more accurately detect constituent chemicals in the return light.

42. The system of claim 41 wherein the polarized UV laser source comprises: (a) an intrinsically polarized laser or (b) another polarizing element in the hand-held instrument and external of the laser source which can be set to one polarization state or optionally adjusted between at least two different polarization states.

43. The system of claim 41 further comprising a battery power source operatively connected to at least one of the laser source, the spectrometer, and the portable computer.
